(19) United States
(12) Patent Application Publication
(10) Pub. No.: US 2025/0312028 A1
(43) Pub. Date: Oct. 9, 2025

(54) INSERTER WITH INTEGRATED CUTTER AND METHOD OF USE

(71) Applicant: Arthrex, Inc., Naples, FL (US)

(72) Inventors: Peter J. Dreyfuss, Naples, FL (US); Joshua S. Dines, Southhampton, NY (US); Brian J. Cole, Chicago, IL (US); Neil S. ElAttrache, Los Angeles, CA (US)

(21) Appl. No.: 19/173,405

(22) Filed: Apr. 8, 2025

Related U.S. Application Data
(60) Provisional application No. 63/631,085, filed on Apr. 8, 2024.

(51) Int. Cl. A61B 17/04; A61F 2/08

(52) U.S. Cl. CPC A61B 17/0401; A61F 2/0811; A61B 2017/0409; A61B 2017/0414; A61B 2017/0464; A61F 2002/0852; A61F 2002/0882

(57) ABSTRACT

Surgical inserters and methods for tissue repair are disclosed. The inserter for tissue repair includes a tube and a shaft, where the shaft is designed to be inserted into a bone hole together an anchor loaded thereon. The inserter also features an integrated cutter that can cut a repair strand used for the repair of damaged tissue.

### CROSS-REFERENCE TO RELATED APPLICATIONS

This application claims priority to U.S. Provisional Patent Application No. 63/631,085, filed Apr. 8, 2024, the entirety of which is hereby incorporated herein by reference.

### TECHNICAL FIELD

This disclosure relates generally to surgical inserters and more particularly to anchor inserters with integrated suture cutters and methods of use for surgical tissue repair.

### BACKGROUND

When soft tissue, such as a ligament or a tendon, becomes detached from a bone, surgery is usually required to reattach the tissue. Typically, a suture anchor, with an attached repair suture, is inserted and secured into a hole in the bone. The repair suture is used to reattach the tissue to bone. U.S. Pat. Nos. 6,544,281, 7,329,272, and 9,005,246 (each commonly owned with the present application and each being incorporated herein by reference in their entirety) provide examples of known suture anchor inserters. Once the suture anchor is inserted and the tissue is fixed to the bone, the surgeon must remove the inserter to conduct post repair procedures, such as cutting of extraneous or leftover suture length. For example, after removal of the inserter from the anchor site, the surgeon then must use a different instrument for cutting the ends of the sutures used in connection with the surgical repair, which adds additional steps to the repair procedure.

### BRIEF SUMMARY

To reduce the steps and time associated with the repair procedure, there exists a need for an integrated inserter that can both insert the anchor and cut the end of the suture. The integrated cutter should be durable, reliable, safe, and easy to use. These needs are met by the inserters and methods of tissue repair disclose herein.

One aspect is directed to an inserter for tissue repair. The inserter includes a tube. The inserter also includes a shaft received within the tube, the shaft being configured to be loaded with an anchor and configured to be inserted into a bone hole. The inserter also includes a cutter configured to cut a repair strand.

Implementations may include one or more of the following features. The inserter can include a handle, and the shaft is configurated to move with respect to the tube and the handle. The tube is immovably fixed to the handle. The inserter can include a lock that is configured to transition between a locked configuration and an unlocked configuration. The lock, in the locked configuration, restricts proximal movement of the shaft relative to the tube by a greater degree than when the lock is in the unlocked configuration. The lock may include a knob that is rotatable relative to the handle, and rotation of the knob transitions the lock between the locked configuration and the unlocked configuration. The lock may include an insert that is received within the handle and that is rotatable with respect to the handle, and the insert is connected the knob and rotatable together with the knob. The insert may include a block and a channel. The block, when the lock is in the locked configuration, is aligned with a proximal end of the shaft to restrict proximal movement of the shaft relative to the tube, and the channel, when the lock is in the unlocked configuration, is aligned with the proximal end of the shaft to permit the proximal end of the shaft to move proximally beyond the block. The tube is a first tube, and the cutter can include a second tube with a sharp at a distal end of the second tube. The cutter can include a guide movably received within the second tube, and the guide is configured to direct the repair strand to the sharp to cut the repair strand. The guide defines a first opening and a second opening, and the guide, when the repair strand is threaded through the first opening and the second opening, secures the repair strand. The inserter can include a retractor that is configured to retract the guide relative to the second tube. The first tube and the second tube are immovably fixed together or are integral, and a distal end of the second tube is proximal to a distal end of the first tube. The inserter may be part of a surgical repair system, which includes the anchor loaded on the shaft. The shaft extends through a hole of the anchor, and an outer diameter of the shaft is larger than an inner diameter of the hole of the anchor. The surgical repair system can include an implant loaded on the shaft distally with respect to the anchor and a repair strand threaded through the implant.

Another general aspect includes a method of tissue repair. The method includes securing a repair strand, captured by an implant loaded on an inserter, to tissue. The method also includes anchoring an anchor, loaded on the inserter, into a bone hole. The method also includes cutting the repair strand, after anchoring the anchor, with a cutter of the inserter.

Implementations may include one or more of the following features. The method where cutting the repair strand can include proximally retracting a guide of the inserter to guide the repair strand into a sharp of the cutter. Anchoring the anchor can include applying a force to a tube of the anchor to drive the anchor into the bone hole. Anchoring the anchor can include, prior to applying the force to the tube, setting the anchor in an opening of the bone hole with a lock of the inserter in a locked configuration. Anchoring the anchor further can include, prior to applying the force to the tube and after setting the anchor in the opening of the bone hole, unlocking the lock to permit a shaft of the inserter to retract proximally as the anchor is driven into the bone hole.

Various additional features and advantages of this invention will become apparent to those of ordinary skill in the art upon review of the following detailed description of the illustrative embodiments taken in conjunction with the accompanying drawings.

### BRIEF DESCRIPTION OF THE SEVERAL VIEWS OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 illustrates a perspective view of aspects of a surgical repair system with a first embodiment of an inserter that includes an integrated cutter according to aspects of this disclosure.

FIG. 2 illustrates an exploded view of the first embodiment of the inserter.

FIG. 3 illustrates a magnified perspective view of aspects of the surgical repair system with the first embodiment of the inserter loaded with an anchor and an implant.

FIG. 4 illustrates a further magnification of the view of FIG. 3 focusing on the cutter of the first embodiment of the inserter.

FIG. 5 illustrates the view of FIG. 4 with a first tube of the inserter removed to isolate a view of a sharp of the inserter.

FIG. 6 illustrates a cross-section view of aspects of the surgical repair system with the first embodiment of the inserter.

FIG. 7 illustrates a perspective view of aspects of a surgical repair system with a second embodiment of the inserter.

FIG. 8 illustrates an exploded view of the second embodiment of the inserter.

FIG. 9 illustrates aspects of the surgical repair system with the second embodiment of the inserter in a first configuration.

FIG. 10 illustrates aspects of the surgical repair system with the second embodiment of the inserter in a second configuration.

FIG. 11 illustrates aspects of the surgical repair system with the second embodiment of the inserter in a third configuration.

FIG. 12 illustrates aspects of the surgical repair system with the second embodiment of the inserter in a fourth configuration.

FIG. 13 illustrates aspects of a surgical repair system with a third embodiment of the inserter.

FIG. 14 illustrates a method of tissue repair.

FIG. 15 illustrates a view of the first embodiment of the inserter in a first configuration.

FIG. 16 illustrates a view of the first embodiment of the inserter in a second configuration.

FIG. 17 illustrates a view of the first embodiment of the inserter in a third configuration.

### DETAILED DESCRIPTION

It is to be understood that the figures and descriptions of the present disclosure may have been simplified to illustrate elements that are relevant for a clear understanding of the present disclosure, while eliminating, for purposes of clarity, other elements found in a typical inserters. Those of ordinary skill in the art will recognize that other elements may be desirable and/or required in order to implement the present disclosure. However, because such elements are well known in the art, and because they do not facilitate a better understanding of the present disclosure, a discussion of such elements is not provided herein. It is also to be understood that the drawings included herewith only provide diagrammatic representations of the presently preferred structures of the present disclosure and that structures falling within the scope of the present disclosure may include structures different than those shown in the drawings. Reference will now be made to the drawings wherein like structures are provided with like reference designations.

Before explaining aspects of the disclosure in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting. It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

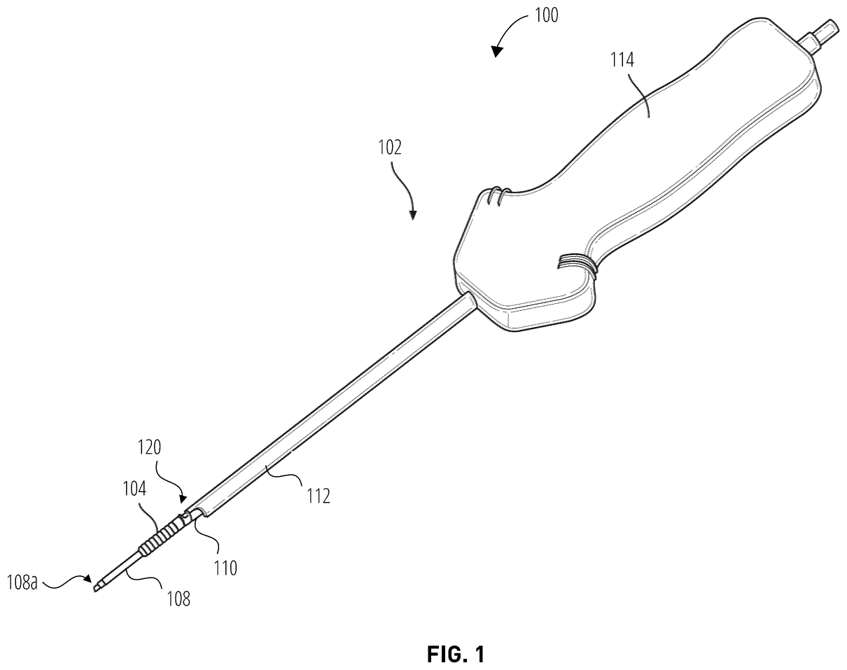

The present disclosure is directed to surgical repair systems, surgical inserters, and methods of using the inserters for surgical tissue repair procedures. In one aspect, an inserter for tissue repair, the inserter includes a tube and a shaft received within the tube. The shaft is configured to be loaded with an anchor and configured to be inserted into a bone hole. The inserter further includes a cutter configured to cut a repair strand. By integrating the cutter with the inserter, time can be saved and surgical repair can be simplified by obviating the need for multiple devices. Moreover, the inserter with the integrated cutter can reduce costs and complexity associated with surgical procedures since the inserter can perform functions that are typically performed by multiple devices. These and other aspects of this disclosure are shown in FIG. 1 through FIG. 17 and are described as follows.

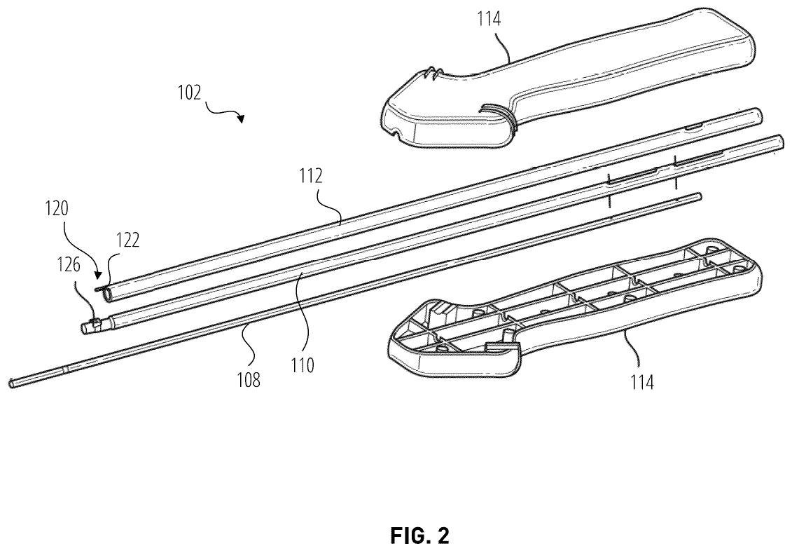

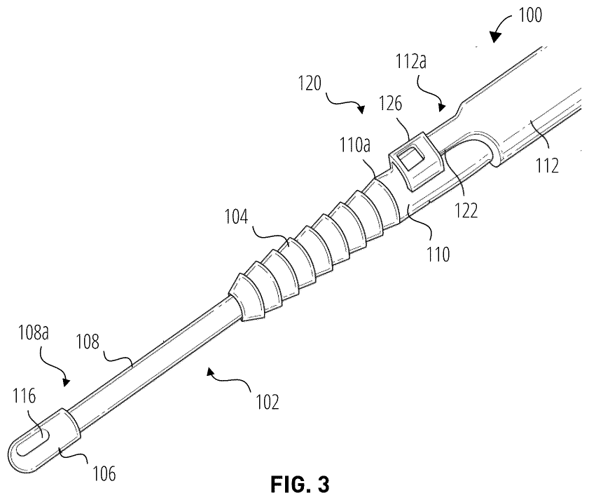

FIG. 1 illustrates a perspective view a surgical repair system 100 with an inserter 102 according to aspects of this disclosure. FIG. 2 shows an exploded view of the inserter 102. FIG. 3 shows a magnified view of a distal end of the inserter 102 that is loaded with an anchor 104 and an implant 106.

The inserter 102 can include a shaft 108. In some implementations, the shaft 108 can comprise a wire, a rod, among other possibilities. The shaft 108 can support an anchor 104 for insertion into a bone hole, as described further subsequently. The inserter 102 can further include a first tube 110. The first tube 110 can drive the anchor 104 into the bone hole, as described further subsequently. In some implementations, the first tube 110 can be an anchor insertion tube, though other implementations are possible. The first tube 110 can receive the shaft 108 therein. For example, an outer diameter of the shaft 108 can be less than an inner diameter of the first tube 110 such that the shaft 108 can be received within the first tube 110. The first tube 110 can be movable relative to the shaft 108. For example, the first tube 110 can move distally and/or proximally relative to the shaft 108.

The inserter 102 can further include a second tube 112. The second tube 112 can support aspects of a cutter 120, which can cut a repair strand 118, as described further subsequently. In some implementations, the second tube 112 can be a cutter tube, though other implementations are possible. The second tube 112 can receive the first tube 110 therein. That is, an outer diameter of the first tube 110 can be less than an inner diameter of the second tube 112 such that the shaft 108 can be received within the first tube 110. The first tube 110 and the second tube 112 can be independently movable relative to each other. The inserter 102 can further include a handle 114 that can house portions of the shaft 108, the first tube 110, and the second tube 112. At least the first tube 110 and the second tube 112 can be movable relative to the handle 114. In embodiments, proximal portions of the first tube 110 and/or the second tube 112 can project from the handle 114 to allow a surgeon or clinician to manipulate the first tube 110 and/or the second tube 112 relative to the handle 114.

The surgical repair system 100 can include the anchor 104, which can be loaded onto a distal end 108a of the shaft 108. The distal end 108a of the distal end 108a can be the distal-most end of the inserter 102. The anchor 104 can be any fixation device that can anchor to bone, including a anchor, a plug, an interference screw, among other possibilities. The anchor 104 can include surface features (e.g., ridges, threads, etc.) to facilitate anchoring and gripping of the walls of the bone hole when the anchor 104 is anchored therein. The anchor 104 can include a hole, which can extend entirely through the anchor 104. The anchor 104 can be loaded onto the shaft 108 by passing the shaft 108 through the hole of the anchor 104. In embodiments, an outer diameter of at least a portion of the distal end 108a of the shaft 108 can be slightly greater than an inner diameter of the hole of the anchor 104. According to this configuration, the anchor 104 can be loaded onto the distal end 108a of the shaft 108 via a friction fit, though other techniques for loading the anchor 104 onto the distal end 108a of the shaft 108 are possible. The anchor 104 can be loaded onto the distal end 108a distally beyond a distal end 110a of the first tube 110. As described further later, the anchor 104 can be anchored into the bone hole by moving the first tube 110 distally with respect to the shaft 108, which can remain stationary. This can cause the distal end 110a to abut a proximal end of the anchor 104, which can forcibly direct the anchor 104 distally along the shaft 108 to anchor the anchor 104 into the bone hole.

The surgical repair system 100 can additionally include the implant 106. The implant 106 can define an eyelet 116 therethrough, which can capture one or more repair strands 118 (e.g., suture, suture tape, among other possibilities) and direct the repair strands 118 into the hole of the bone. The implant 106 can be removably loaded (e.g., press fit, threaded, among other possibilities) onto a distal end 108a of the shaft 108, as shown in FIG. 3. In embodiments, once the anchor 104 is anchored into the bone hole, the inserter 102 can be removed and the anchor 104 can cause the implant 106 to disengage from the distal end 108a of the shaft 108 such that the implant 106 remains in the bone hole with the anchor 104. In embodiments, the anchor 104 can engage with the implant 106 within the bone hole when anchored therein.

The inserter 102 can further include a cutter 120, which can cut the repair strand 118. By integrating the cutter 120 into the inserter 102, the inserter 102 can both insert the anchor 104 and/or implant 106 and trim or cut the repair strand 118. This can save time and simplify surgical repair by obviating the need for multiple devices. Moreover, this can reduce costs and complexity associated with surgical procedures since the inserter 102 can perform functions that are typically performed by multiple devices.

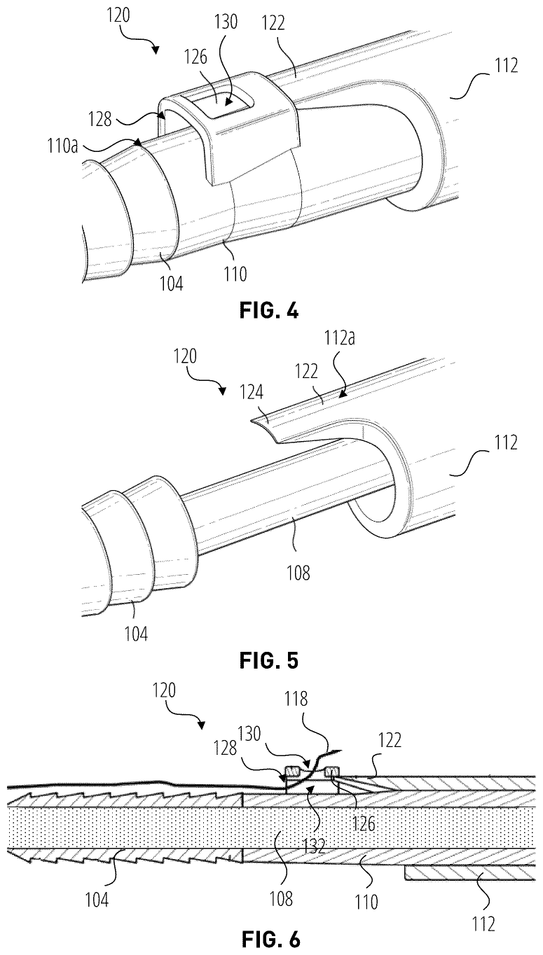

FIG. 4 shows a further magnification of the view of FIG. 3, focusing on aspects of the cutter 120. FIG. 5 shows the view of FIG. 4 with the first tube 110 removed to isolate a sharp 122 of the cutter 120. FIG. 6 illustrates a cross-section view of aspects of the cutter 120. In embodiments, the first tube 110 and the second tube 112 can each include aspects of the cutter 120. For example and as shown best in FIG. 5, a distal end 112a of the second tube 112 can include a sharp 122 with an edge 124 that can cut the repair strand 118. As shown best in FIG. 4, the first tube 110 can include a guide 126 that can cooperate with the sharp 122 to guide the repair strand 118 to the edge 124 to cause the edge 124 to cut the repair strand 118. The guide 126 can be located at or adjacent to the distal end 110a of the first tube 110. The guide 126 can be arranged at or on an outer surface of the first tube 110. The guide 126 can define a first opening 128 and a second opening 130. As best shown in FIG. 6, the repair strand 118 can be threaded through the first opening 128 and the second opening 130 of the guide 126 before the repair strand 118 is cut. To cut the repair strand 118, the second tube 112 can be moved distally from the position shown in FIG. 6 and relative to the first tube 110, which can drive the sharp 122 into an interior 132 of the guide 126 and cause the edge 124 to cut the repair strand 118.

In an aspect, the inserter 102 can be prepackaged with the anchor 104 and the implant 106 loaded on the shaft 108. For example, the surgical repair system 100 can be packaged together in a kit with the anchor 104 and the implant 106 loaded on the shaft 108. The inserter 102 can also be prepackaged with the repair strand 118 threaded through the eyelet 116 of the implant 106 and through the first opening 128 and the second opening 130 of the guide 126. This can be advantageous in that the inserter 102 can be in a ready to use state, which can reduce complexity and save time during surgical repair procedures.

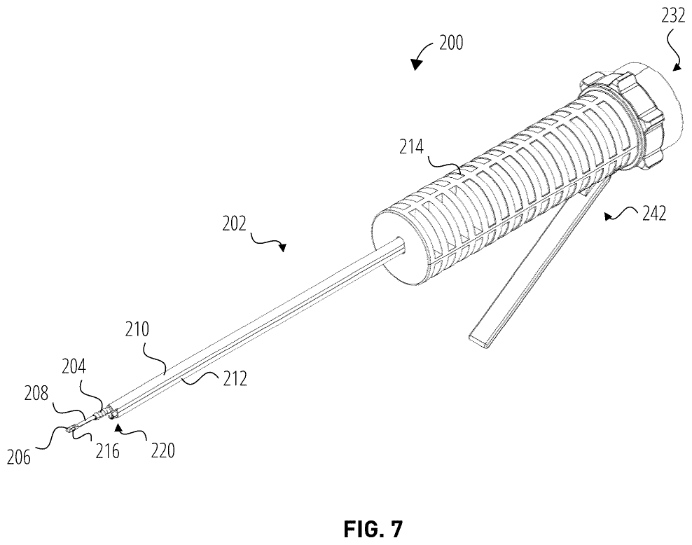

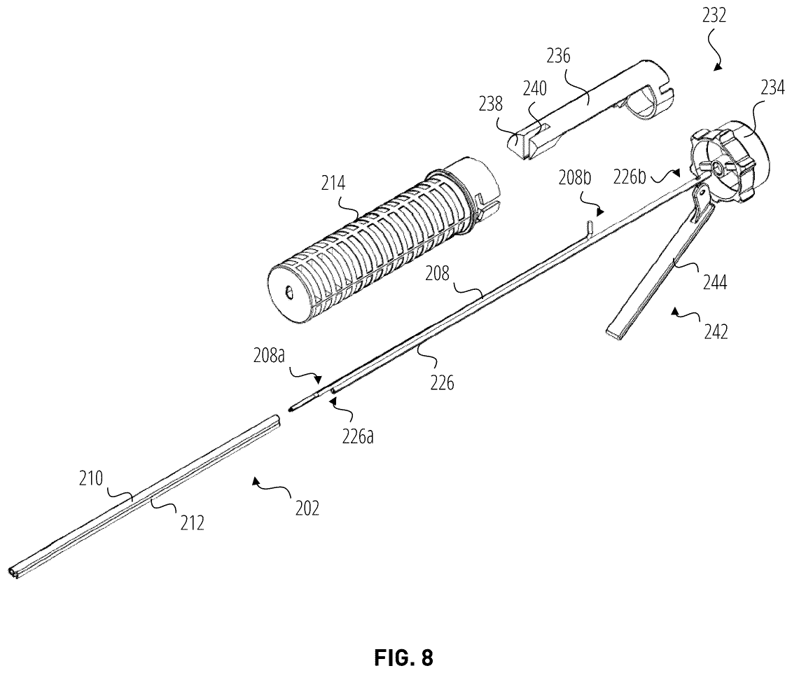

FIG. 7 through FIG. 12 show aspects of a surgical repair system 200 with an inserter 202 according to aspects of this disclosure. FIG. 7 shows a perspective view of the surgical repair system 200. FIG. 8 shows an exploded view of the inserter 202. Except for mutually exclusive aspects and except where explicitly described to the contrary, the surgical repair system 200 and the inserter 202 can respectively include any of the aspects described previously with respect to the surgical repair system 100 and the inserter 102, and vice versa.

The inserter 202 can include a shaft 208. The shaft 208 can support an anchor 204 for insertion into a bone hole, as described further subsequently. In some implementations, the shaft 208 can comprise a wire, a rod, among other possibilities. The inserter 202 can further include a first tube 210. The first tube 210 can drive the anchor 204 into the bone hole, as described further subsequently. In some implementations, the first tube 210 can be an anchor insertion tube, though other implementations are possible. The first tube 210 can receive the shaft 208 therein. An outer diameter of the shaft 208 can be less than an inner diameter of the first tube 210 and the shaft 208 can be received within the first tube 210. The shaft 208 can be movable relative to the first tube 210. For example, the shaft 208 can move distally and/or proximally relative to the first tube 210. In embodiments, the first tube 210 can be immovably fixed relative to the handle 214.

The surgical repair system 200 can include the anchor 204, which can be loaded onto a distal end 208a of the shaft 208. The distal end 208a of the shaft 208 can be the distal-most end of the inserter 202. The anchor 204 can be any structure that can anchor to bone, including a plug, an interference screw, among other possibilities. The anchor 204 can include surface features (e.g., ridges, threads, etc.) to facilitate anchoring and gripping of the walls of the bone hole when the anchor 204 is inserted therein. The anchor 204 can include a hole, which can extend entirely through the anchor 204. The anchor 204 can be loaded onto the shaft 208 by passing the shaft 208 through the hole of the anchor 204. In embodiments, an outer diameter of at least a portion of the distal end 208a of the shaft 208 (e.g., a portion proximal to the region 208c described later) can be slightly greater than an inner diameter of the hole of the anchor 204. According to this configuration, the anchor 204 can be loaded onto the distal end 208a of the shaft 208 via a friction fit, though other techniques for loading the anchor 204 onto the distal end 208a of the shaft 208 are possible. The anchor 204 can be loaded onto the distal end 208a distally beyond a distal end 210a of the first tube 210. As described further later, the anchor 204 can be anchored into the bone hole by moving the first tube 210 distally, such as by applying a force to the handle 214 to drive the first tube 210 immovably fixed thereto distally. This can cause the distal end 210a to abut a proximal end of the anchor 204, which can forcibly direct the anchor 204 distally along the shaft 208 to anchor the anchor 204 into the bone hole.

The surgical repair system 200 can additionally include the implant 206. The implant 206 can define an eyelet 216 therethrough, which can capture one or more repair strands 218 (e.g., suture, suture tape, among other possibilities) and direct the repair strands 218 into the hole of the bone. The implant 206 can be removably loaded (e.g., press fit, threaded, among other possibilities) onto the distal end 208a of the shaft 208, as shown in FIG. 7. In embodiments, once the anchor 204 is anchored into the bone hole, the inserter 202 can be removed and the anchor 204 can cause the implant 206 to disengage from the distal end 208a of the shaft 208 such that the implant 206 remains in the bone hole with the anchor 204. In embodiments, the anchor 204 can engage with the implant 206 within the bone hole.

The inserter 202 can further include a lock 232. In a locked configuration, the lock 232 can restrict proximal movement of the shaft 208 relative to the first tube 210 by a greater degree than when the lock 232 is in the unlocked configuration. In embodiments, the lock 232 can prevent any proximal movement of the shaft 208 relative to the first tube 210. In an unlocked configuration, the lock 232 can permit proximal movement of the shaft 208 relative to the first tube 210 by a greater degree than when the lock 232 is in the locked configuration. As shown in FIG. 8, the lock 232 can include a knob 234 and an insert 236 that can rotate together, relative to the handle 214, about a longitudinal axis of the inserter 202. Rotating the knob 234 and the insert 236 can transition the lock 232 between the locked configuration and the unlocked configuration. The knob 234 and the insert 236 can be distinct or integral components. The insert 236 can be rotatably received within an interior of the handle 214. The knob 234 can be rotatably connected to a proximal end of the handle 214.

The insert 236 can include a stop 238. In the locked configuration, the stop 238 can align with the proximal end 208b of the shaft 208 and can limit or entirely prevent proximal movement of the shaft 208. This, in turn, can lock the shaft 208 and can restrict or prevent the shaft 208 from moving proximally relative to the first tube 210.

The insert 236 can include a channel 240. In the unlocked configuration, the stop 238 can be misaligned with the proximal end 208b of the shaft 208 and the proximal end 208b can be aligned with the channel 240. This can allow proximal movement of the proximal end 208b of the shaft 208 within the channel 240 and thus can allow the shaft 208 to move proximally relative to the first tube 210. In embodiments, the channel 240 can limit proximal movement of the shaft 208 to a predetermined range. For example, the channel 240 can have a fixed length in the proximal direction and proximal movement of the shaft 208 can be limited to the fixed length when the lock 232 is in the unlocked configuration.

The inserter 202 can further include a cutter 220, which can cut the repair strand 218. By integrating the cutter 220 with the inserter 202, the inserter 202 can both insert the anchor 204 and/or implant 206 and trim or cut the repair strand 218. This can save time and simplify surgical repair by obviating the need for multiple devices. Moreover, this can reduce costs and complexity associated with surgical procedures since the inserter 202 can perform functions that are typically performed by multiple devices.

In embodiments, the cutter 220 can include a second tube 212. In some implementations, the second tube 212 can be a cutter tube, though other implementations are possible. The second tube 212 can include a sharp 222 having an edge 224 for cutting repair strands 218. The sharp 222 can be at the distal end 212a of the second tube 212. In embodiments, the second tube 212 can be immovably fixed relative to the handle 214 and/or relative to the first tube 210. In embodiments, the second tube 212 can fixed to, or integral with, the first tube 210. In embodiments, the second tube 212 and the first tube 210 can be arranged in a tangential, or side-by-side, relationship. The distal end 212a of the second tube 212 can be fixed proximally away from the distal end 210a of the first tube 210. This can be advantageous in that the distal end 212a does not interfere with placement of the anchor 204, which as previously described, abuts the distal end 210a of the first tube 210 when driven into the bone hole.

The cutter 220 can further include a guide 226 that can guide the repair strand 218 to the sharp 222 to cut the repair strands 218. The guide 226 can be partially or entirely tubular and can be received within the second tube 212. An outer diameter of the guide 226 can be less than an inner diameter of the second tube 212 such that the guide 226 can be received therein. The guide 226 can move proximally and/or distally relative to the second tube 212 and relative to the handle 214. The guide 226 can define a first opening 228 and a second opening 230 at a distal end 226a thereof.

The cutter 220 can further include a retractor 242 operatively connected to a proximal end 226b of the guide 226. Actuation of the retractor 242 can cause the guide 226 to move proximally with respect to the second tube 212. As described further later, when a repair strand 218 is threaded through the first opening 228 and the second opening 230 and the retractor 242 is actuated, the guide 226 can move proximally, which can in turn force the repair strand 218 against the edges 224 of the sharp 222 to cut the repair strand 218. In embodiments, the retractor 242 can include a retractor handle 244 that can be connected to the proximal end 226b of the guide 226 and that can be movably connected (e.g., hingedly connected) to the handle 214. In such embodiments, actuation of the retractor 242 can involve moving the retractor handle 244 towards the handle 214 by pressing or squeezing the retractor handle 244.

In an aspect, the inserter 202 can be prepackaged in a kit with the anchor 204 and the implant 206 loaded on the shaft 208. The kit can also be prepackaged with the repair strand 218 threaded through the eyelet 216 of the implant 206 and through the first opening 228 and the second opening 230 of the guide 226. This can be advantageous in that the inserter 202 can be in a ready to use state, which can reduce complexity and save time during surgical repair procedures.

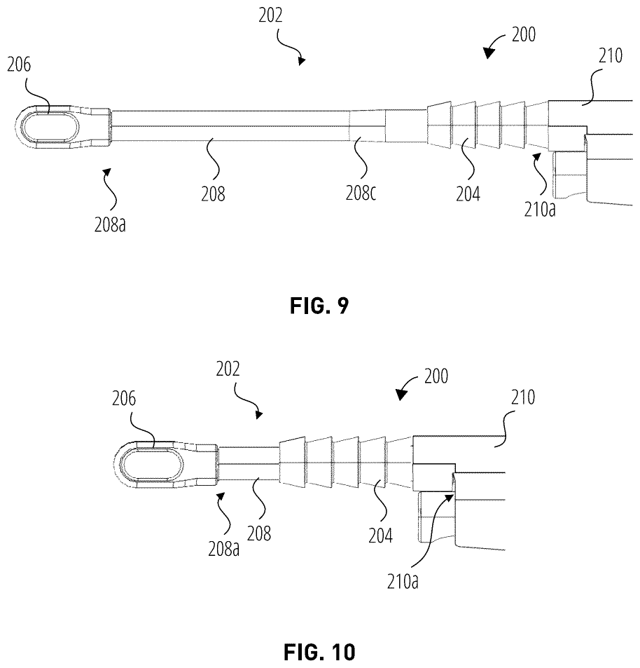

FIG. 9 shows aspects of the surgical repair system 200 including a magnified view of a distal end of the inserter 202 with the anchor 204 loaded onto the shaft 208. In embodiments, such as shown in FIG. 9, the shaft 208 can include a region 208c that tapers towards the distal end 208a. This can be advantageous in that the anchor 204 can be loaded, at least partially, proximal to the region 208c to securely hold the anchor 204 during insertion into the bone hole. In alternative embodiments, the shaft 208 can be provided without the region 208c and the diameter of the distal end 208a can be constant. When the lock 232 is in the first configuration and force is applied to the handle 214, the shaft 208 and the first tube 210 can be inserted together into the patient and the lock 232 can prevent proximal movement of the shaft 208 relative to the first tube 210. This can allow the surgeon to set the implant 206 (and repair strands threaded thereto) into an opening of the bone hole via the handle 214.

FIG. 10 shows a magnified view of the distal end of the inserter 202 with distal end 208a of the shaft 208 driven distally towards the distal end 210a of the first tube 210. As previously described, when the lock 232 is in the unlocked configuration the shaft 208 can move proximally relative to the first tube 210, which in embodiments can remain stationary relative to the handle 214. This can cause the anchor 204 to abut against the distal end 210a of the first tube 210 and can cause the shaft 208 to retract proximally relative to the anchor 204 and close the distance between the implant 206 and the anchor 204. For example, force can be applied to the handle 214 to drive the first tube 210 towards the bone hole. Since the shaft 208 can retract proximally relative to the first tube 210 when the lock 232 is in the second configuration, this force can be directed to the anchor 204 to anchor the anchor 204 into the bone hole. The shaft 208 can concurrently retract proximally within and relative to the first tube 210 to reduce or eliminate the transmission of distal force into the shaft 208 and thereby prevent further penetration of the shaft 208 into the bone hole. In embodiments with the region 208c, retraction of the shaft 208 can move the anchor 204 partially or entirely distally beyond the region 208c to a region of the shaft 208 with a smaller diameter. This can facilitate removal of the anchor 204 and reduce the risk that friction between the shaft 208 and the anchor 204 dislodges the anchor 204 upon removal.

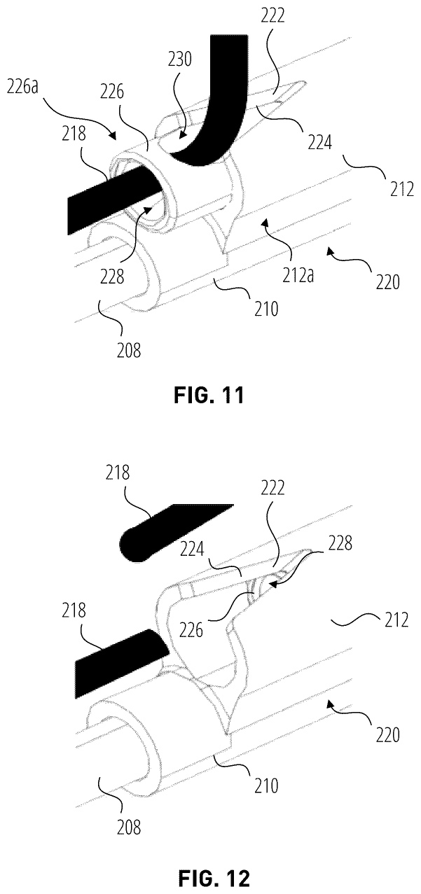

FIG. 11 shows a magnified view of the cutter 220 of the inserter 202 pre-cut configuration. The repair strand 218 can be threaded through the first opening 228 and the second opening 230. In embodiments, before the retractor 242 is actuated the guide 226 can hold the repair strand 218 distally away from the edge 224 to prevent premature cutting of the repair strand 218.

FIG. 12 shows a magnified view of the cutter 220 of the inserter 202 in post-cut configuration after the repair strand 218 is cut. The inserter 202 can transition from the pre-cut configuration (shown in FIG. 11) to the post-cut configuration (shown in FIG. 12) upon actuation of the retractor 242. As previously described, actuation of the retractor 242 can cause the guide 226 to retract proximally relative to the second tube 212. When the repair strand 218 is threaded through the first opening 228 and the second opening 230, this retraction can force the repair strand 218 against the cutting edge 224 to sever the repair strand 218.

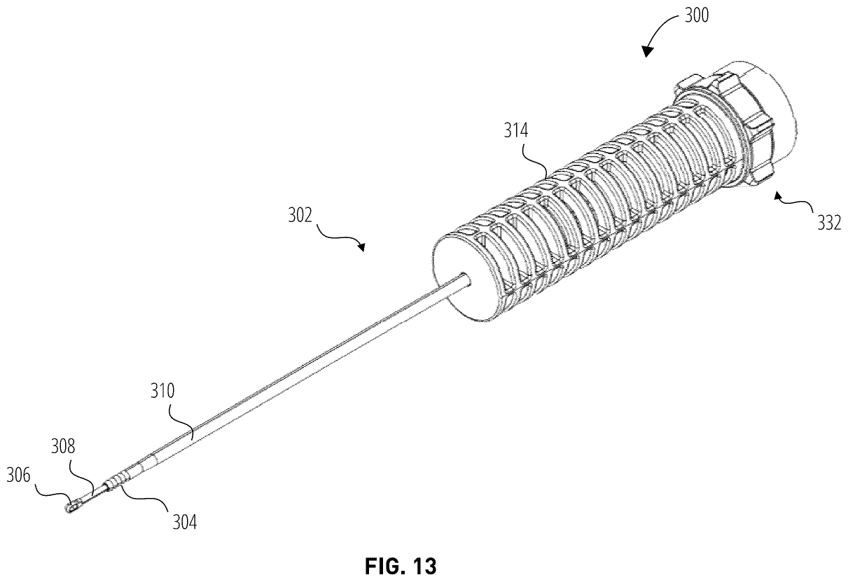

FIG. 13 shows a perspective view of a surgical repair system 300 with an inserter 302. Except for mutually exclusive aspects and except where explicitly described to the contrary, the surgical repair system 300 and the inserter 302 can respectively include any of the aspects described previously with respect to the surgical repair system 200 and the inserter 202. For example, the inserter 302 can be loaded with an anchor 304 and/or an implant 306. The inserter 302 can include a shaft 308 that can be movable relative to a first tube 310, which can be fixed with respect to a handle 314. The inserter 302 can further include a lock 332 that can selectively lock the shaft 308 relative to the first tube 310, as previously described. The inserter 302 can be provided without an integral cutter. The inserter 302 can be advantageous over known inserters even without the cutter at least because of the structural relationship between the shaft 308, which is selectively moveable based upon a configuration of the lock 332, and the first tube 310, which can be fixed relative to the handle 314, as previously described.

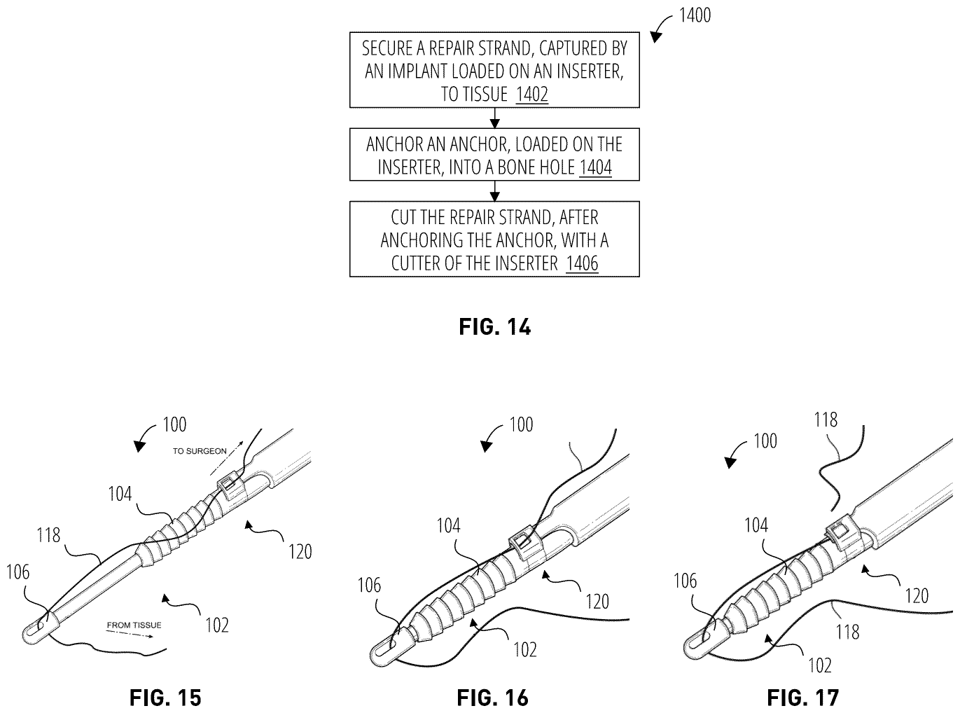

FIG. 14 shows a method of tissue repair 1400 according to aspects of this disclosure. The method of tissue repair 1400 can be performed with the surgical repair system 100 having the inserter 102 or with the surgical repair system 200 having the inserter 202.

In step 1402, the method of tissue repair 1400 can including securing a repair strand, captured by an implant loaded on an inserter, to tissue. FIG. 15 shows a view of the surgical repair system 100 with the implant 106 loaded on the inserter 102 and the repair strand 118 threaded therethrough. It is to be understood that the surgical repair system 200 can be analogously arranged.

In step 1404, the method of tissue repair 1400 can include anchoring an anchor, loaded on the inserter, into a bone hole. FIG. 16 shows a view of the surgical repair system 100 in which the inserter 102 drives the anchor 104 and the implant 106 together, as would occur within a bone hole in step 1404. It is to be understood that the surgical repair system 200 can be analogously arranged. For example and as previously described, the second tube 212 of the inserter 202 can drive the anchor 204 into the bone hole. This can occur, for example, when a force (e.g., a mallet strike) is applied to the handle 214 which can in turn translate the force to the second tube 212 to drive the anchor 204 into the bone hole. Prior to application of the force, the lock 232 can be unlocked to permit the shaft 208 to move proximally with respect to the second tube 212 and prevent the shaft 208 from being over inserted into the bone hole. In embodiments, the lock 232 can be in the locked configuration when the anchor 204 is initially inserted into the bone hole but before the first tube 210 drives the anchor 204. As previously described, this can restrict proximal movement of the shaft 208 with respect to the first tube 210, which can give the surgeon or clinician greater control when setting the anchor 204 in the opening of the bone hole.

In step 1406, the method of tissue repair 1400 can include cutting the repair strand, after anchoring the anchor, with a cutter of the inserter. FIG. 17 shows a view of the surgical repair system 100 with the repair strand 118 cut by the cutter 120 of the inserter 102. It is to be understood that the surgical repair system 200 can be analogously arranged. For example and as previously described, the retractor 242 can cause the guide 226 to move proximally with respect to the second tube 212 to cut the repair strand 218, as shown in FIG. 12.

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

What is claimed is:

1. An inserter for tissue repair, the inserter comprising:
a tube;
a shaft received within the tube, the shaft being configured to be loaded with an anchor and configured to be inserted into a bone hole; and
a cutter configured to cut a repair strand.

2. The inserter of claim 1, wherein:
the inserter further comprises a handle, and
the shaft is configurated to move with respect to the tube and the handle.

3. The inserter of claim 2, wherein the tube is immovably fixed to the handle.

4. The inserter of claim 2, wherein:
the inserter further comprises a lock that is configured to transition between a locked configuration and an unlocked configuration, and
the lock, in the locked configuration, restricts proximal movement of the shaft relative to the tube by a greater degree than when the lock is in the unlocked configuration.

5. The inserter of claim 4, wherein:
the lock comprises a knob that is rotatable relative to the handle, and
rotation of the knob transitions the lock between the locked configuration and the unlocked configuration.

6. The inserter of claim 5, wherein:
the lock comprises an insert that is received within the handle and that is rotatable with respect to the handle, and
the insert is connected the knob and rotatable together with the knob.

7. The inserter of claim 6, wherein the insert comprises a block and a channel.

8. The inserter of claim 7, wherein:
the block, when the lock is in the locked configuration, is aligned with a proximal end of the shaft to restrict proximal movement of the shaft relative to the tube, and
the channel, when the lock is in the unlocked configuration, is aligned with the proximal end of the shaft to permit the proximal end of the shaft to move proximally beyond the block.

9. The inserter of claim 1, wherein:
the tube is a first tube,
the cutter comprises a second tube comprising a sharp at a distal end of the second tube,
the cutter further comprises a guide movably received within the second tube, and
the guide is configured to direct the repair strand to the sharp to cut the repair strand.

10. The inserter of claim 9, wherein:
the guide defines a first opening and a second opening, and
the guide, when the repair strand is threaded through the first opening and the second opening, secures the repair strand.

11. The inserter of claim 9, further comprising a retractor that is configured to retract the guide relative to the second tube.

12. The inserter of claim 9, wherein:
the first tube and the second tube are immovably fixed together or are integral, and
a distal end of the second tube is proximal to a distal end of the first tube.

13. A surgical repair system comprising:
the inserter of claim 1; and
the anchor loaded on the shaft.

14. The surgical repair system of claim 13, wherein:
the shaft extends through a hole of the anchor, and
an outer diameter of the shaft is larger than an inner diameter of the hole of the anchor.

15. The surgical repair system of claim 13, further comprising:
an implant loaded on the shaft distally with respect to the anchor; and
a repair strand threaded through the implant.

16. A method of tissue repair comprising:
securing a repair strand, captured by an implant loaded on an inserter, to tissue;
anchoring an anchor, loaded on the inserter, into a bone hole; and
cutting the repair strand, after anchoring the anchor, with a cutter of the inserter.

17. The method of claim 16, wherein cutting the repair strand comprises proximally retracting a guide of the inserter to guide the repair strand into a sharp of the cutter.

18. The method of claim 16, wherein anchoring the anchor comprises applying a force to a tube of the anchor to drive the anchor into the bone hole.

19. The method of claim 18, wherein anchoring the anchor further comprises, prior to applying the force to the tube, setting the anchor in an opening of the bone hole with a lock of the inserter in a locked configuration.

20. The method of claim 19, wherein anchoring the anchor further comprises, prior to applying the force to the tube and after setting the anchor in the opening of the bone hole, unlocking the lock to permit a shaft of the inserter to retract proximally as the anchor is driven into the bone hole.
